(19) United States
(12) Patent Application Publication
(10) Pub. No.: US 2019/0320997 A1
(43) Pub. Date: Oct. 24, 2019

(54) METHODS FOR THE USE OF INHERENT FREQUENCY SHIFTING MECHANISMS FOR SENSORS RESPONSE READING WITH CONTINUOUS WAVE EXCITATION

(71) Applicant: Microtech Medical Technologies Ltd., Tel Aviv (IL)

(72) Inventors: Amir KOHEN, Tel Aviv (IL); Yuval BECKER, Tel Aviv (IL); Eric S. TAMMAM, Modiin (IL)

(21) Appl. No.: 16/389,202

(22) Filed: Apr. 19, 2019

Related U.S. Application Data
(60) Provisional application No. 62/661,925, filed on Apr. 24, 2018.

(51) Int. Cl. A61B 8/04; G01L 11/06; G01H 13/00; A61B 8/12

(52) U.S. Cl. CPC A61B 8/04; A61B 8/12; G01H 13/00; G01L 11/06

(57) ABSTRACT

A method and system of the invention generally relate to measuring ambient pressure in systems comprising incompressible fluids. Particularly, the method and system relate to monitoring pressure within body lumens. The ambient pressure may be measured by transmitting a frequency comb having non-uniform spacing between transmitted frequencies at the passive sensor and measuring the frequency response of the passive sensor. In one embodiment, a higher-order harmonic of the sensor is excited and measured to determine the ambient pressure. In another embodiment, the frequency response of frequencies in-between the transmitted frequencies are measured to determine the ambient pressure.

This application claims the benefit of priority of U.S. Provisional Application Ser. No. 62/661,925 filed Apr. 24, 2018, which is incorporated by reference in its entirety.

### FIELD OF INVENTION

The invention generally relates a method and system for measuring ambient pressure in systems comprising incompressible fluids. Particularly, the method and system relate to monitoring pressure within body lumens.

### BACKGROUND

Prior art pressure measurement systems are based on the measurement of the resonant frequency of a passive mechanical resonator, i.e., a sensor. In the standard operation mode such a sensor is excited by a driving force provided by an externally located ultrasonic transducer emitting a sum of ultrasonic signals spanning a predefined frequency range. As a result of this driving force, the sensor will oscillate with an amplitude and phase reflecting its spectral response, maximum amplitude response is expected at the resonance frequency. In the case of a pressure sensor, the resonant frequency of the sensor changes as a function of pressure therefor allowing one to determine the ambient pressure experienced by the sensor by detecting the resonant frequency. However since the energy transmitted for excitation of the sensor is much greater than that of the signal produced by the sensor, the transmitted energy thereby “masks” the sensor's response at the frequencies being transmitted.

There exists a strong clinical need for a pressure monitoring system that can provide accurate pressure measurements of body lumens while allowing the physician to monitor those pressures non-invasively while avoiding such a “masking.”

Prior art devices detect pressure by interrogating a passive pressure sensor at or around the lowest resonant frequency. However, in certain situations (e.g., non-linear systems), the information gained from exciting a first harmonic of the system may not provide enough information to accurately compute the ambient pressure. Thus, a need exists for more robust methods of computing the ambient pressure using the frequency response of a passive pressure sensor.

### SUMMARY OF THE INVENTION

The present invention relates to a method and apparatus for measuring pressures in body lumens. The apparatus is a passive mechanical resonator, which in embodiments may be a sensor device, that is miniature, passive, implantable and wireless, to allow for non-invasive, frequent monitoring of portal venous pressure. The sensor device is miniature to allow for safe implantation into the target vessels. In one embodiment, the sensor device structure comprises a single sensor unit having a sensor membrane of a thickness greater than at least 1 micron and an overall sensor device size range of 0.1 mm-1 mm in width (w), 0.1 mm-1 mm in depth (d), and 0.1 mm-0.75 mm in height (h). The overall volume of the sensor device will preferably not exceed 0.3 cubic millimeters. Other examples of volumetric ranges (in mm³) for the sensor device are, e.g., 0.005-0.008, 0.01-0.09, or 0.1-0.3. The apparatus is passive to allow the treating physician to monitor the patient as often as is desired or needed. The invention is useful for interrogating ambient conditions in systems that comprise an incompressible fluid particularly in measuring portal and/or hepatic pressures.

An object of the invention is to provide a method for measuring body lumen pressure, with an implanted and anchored sensor device in a body lumen comprising the steps of: applying a frequency comb of acoustic waves to the sensor, receiving the frequencies elicited in the sensor by the frequency comb, and processing the received higher harmonics of the applied frequencies as acoustic data in order to determine the frequency response, e.g., resonance frequency, of the vibratable sensor, and thereby determine the ambient fluid pressure of the environment in which the sensor is disposed.

Another object of the invention is to provide a method for measuring ambient fluid pressure in a subject system, from a sensor device disposed in the subject system, where the sensor device includes a vibration sensor with a sensor membrane that has a resonance frequency response and higher order frequency responses, such as second harmonic frequencies, dependent on ambient pressure conditions and a plurality of frequency responses per given pressure, comprising the steps of: subjecting the sensor to a frequency comb of acoustic waves in order to elicit acoustic resonances or vibrations in the sensor, detecting the acoustic resonances as reflected signals from the sensor, and processing the detected acoustic resonances in order to determine ambient fluid pressure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a device in accordance with the invention for measuring portal venous pressure.

FIG. 2 shows a system in accordance with the invention for measuring, interpreting, and displaying portal venous pressure.

FIG. 3 illustrates an example of a linear spectral response as a function of pressure where the different curves each represent a different frequency.

FIG. 4 illustrates an example of a linear spectral pressure response at a second harmonic as a function of pressure.

FIG. 5 illustrates an example of a spectral pressure response at a second harmonic as a function of pressure corrected by using the special comb.

FIGS. 6A-6C illustrate the signal before and after the subtraction of the response at frequencies.

### DETAILED DESCRIPTION OF THE INVENTION

The method and apparatus of the invention generally relate to measuring ambient pressure in a system comprising an incompressible fluid. For purposes of this application, “incompressible fluid” refers generally to non-vapor, non-compressible, flowable media, such as liquids, slurries and gels. The miniature size of the apparatus, compared to current conventional devices for measuring ambient fluid pressure, and relatively low invasiveness of the apparatus and method are particularly well suited to medical and physiological applications, including, but not limited to, measuring: i) blood vessel/artery/vein pressures such as, for example, in portal hypertension; ii) spinal fluid pressure in brain ventricles; iii) intra-abdominal pressures such as in the urinary tract, bladder, kidney, and bile ducts; and the like. The ambient pressure may be measured by transmitting a frequency comb having non-uniform spacing between transmitted frequencies at the passive sensor and measuring the frequency response of the passive sensor. In one embodiment, a higher-order harmonic of the sensor is excited and measured to determine the ambient pressure. In another embodiment, the frequency response of frequencies in-between the transmitted frequencies are measured to determine the ambient pressure. The method may be applicable to any disease or condition involving bodily systems through which fluids, i.e., incompressible fluids, e.g., liquids, flow.

One object of the present invention is to provide a passive mechanical resonator, which in embodiments may be a sensor device, for measuring ambient fluid pressure in a system comprising an incompressible fluid, e.g., a liquid. The sensor device may be a naked vibratable sensor or a vibratable sensor housed in a cavity with or without a bottom film sealing the housing. In one embodiment, the sensor device comprises a vibratable sensor having a sensor membrane, which sensor membrane has a resonance frequency responsive to ambient fluid pressure conditions. The sensor membrane has a thickness in the range of 1 micron-200 microns and forms one side of a chamber. The chamber is defined by the sensor membrane and a plurality of walls which are substantially perpendicular to the sensor membrane. The chamber may be sealed with a compressible gas of predefined pressure disposed therein. The chamber is sealed with a bonding layer using an anodic bonding process. The bonding layer may provide a means for attachment of the vibratable sensor to an anchoring device. As such, the sensor device comprising a naked vibratable sensor may be a hermetically sealed, substantially or partially non-solid component of any shape having a sensor membrane and a chamber. Alternatively, the vibratable sensor may be an acoustically-active solid, i.e., a sensor membrane without a chamber. In either aspect, the vibratable sensor is biocompatible, i.e., substantially non-reactive within a human body.

In another embodiment, the vibratable sensor may be disposed in a cavity defined by a housing. In this embodiment a cover plate covers the housing cavity such that the bonding layer faces the cover plate. A base plate forms the foundation for the housing. The base plate may contain an orifice exposing the sensor membrane of the vibratable sensor to the bodily environment to be measured. In one aspect of this embodiment, the housing further comprises a bottom film. The bottom film may be semi-permeable or non-permeable to external fluids and/or tissues and may enclose an incompressible fluid.

In one embodiment, a sensor device may be implanted in the portal vein thereby providing a combination of hemostatic and intra-abdominal pressure. In another embodiment, a sensor device may be implanted in each of the hepatic and portal venous systems. Implantation into the portal vein may be carried out via a transhepatic puncture using either an intracostal or subxiphoid approach, while the hepatic vein implantation may be carried out through the transjugular approach. In this way, the system may provide information on the pressure gradient between the hepatic venous systems. In this latter embodiment, the system provides both the porto-hepatic pressure gradient and the portal venous pressure in the same session. Implanting the sensor may also include the steps of anchoring the sensor to a bodily tissue or organ, or securing the sensor to a scaffold and implanting the scaffold.

The invention is discussed and explained below with reference to the accompanying drawings. The drawings are provided as an exemplary understanding of the invention and to schematically illustrate particular embodiments and details of the invention. The skilled artisan will readily recognize other similar examples equally within the scope of the invention. The drawings are not intended to limit the scope of the invention as defined in the appended claims.

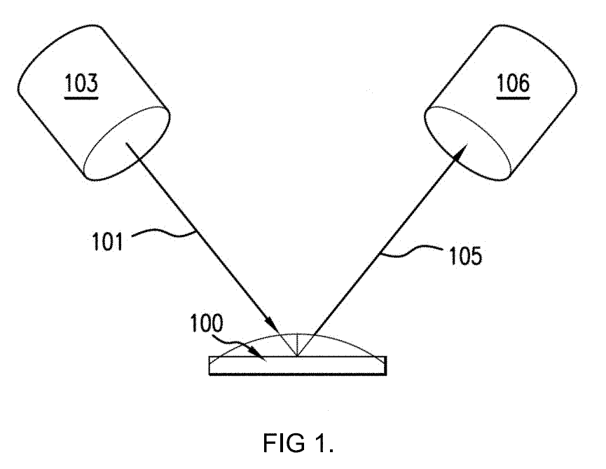

FIG. 1 illustrates a sensor device system of the invention. Sensor device 100 measures ambient pressure of the implanted sensor device. Sensor device 100 is subjected to a frequency comb of acoustic waves 101, which are generated by frequency transmitter 103. As it is used in this application, frequency comb means a collection of acoustic waves at a range of calibrated frequency all transmitted in generally the same direction. In certain embodiments, the frequency comb may have from 3 to 16 waves of different frequencies. Frequency transmitters 103 may comprise any transducer suitable for controllably generating acoustic energy beams (such as, but not limited to sonic or ultrasonic beams) as is known in the art. Typically such transducers are called tactile transducers and are capable of converting an electrical signal into, for example, vibrations that may be felt or used for work. The transducers provide a field of view comprising a depth of penetration of 4-16 cm and a beam spot diameter of 3 cm generating a measurement ellipsoid, for example. The transducers may be implemented using suitable piezoelectric transducers, but other transducers known in the art may be used, such as, but not limited to, capacitive transducers, wideband capacitive transducers, composite piezoelectric transducers, electromagnetic transducers, various transducer array types and various suitable combinations of such transducers configured for obtaining different frequencies and/or beam shapes. For example, acoustic transmitters manufactured by Vemco, PCB Piezoelectronics, and Hardy Instruments may be used. An acoustic wave frequency comb 101 is directed at the sensor device 100, producing modulated acoustic waves 105 that are detected by an ultrasound receiver 106. Subsequent processing of modulated acoustic waves 105 enables calculation of the ambient pressure in device 100. This invention may also be used with any pressure sensor or any non-linear sensor. Other such sensors are RFID sensors and electrical sensors with coils, capacitors, and resistors. Practical electric components never create perfectly linear frequency responses. This non-linearity is heightened when operated at high signal levels. Use of active components such as diodes and transistors has an inherent non-linear effect. Such nonlinear effects lead to the generation of higher harmonics, which may be used with the present invention.

One aspect of the invention relates to an implantable sensor device comprising a miniature sensor device for measuring ambient fluid pressure. The sensor device comprises a vibratable sensor having a sensor membrane, which has a frequency response to ambient pressure conditions. The sensor membrane of the vibratable sensor forms one side of a chamber wherein resides a compressible gas of predefined pressure. The chamber is further defined by at least one wall which is preferably substantially perpendicular to the sensor membrane. In one embodiment, the vibratable sensor is made of silicon, but other suitable materials may be used, for example a metal, Pyrex® or other glass, boron nitride, or the like. Non-limiting examples of metals include, e.g., titanium, gold, stainless steel, platinum, tantalum, or any suitable metal, alloy, shape memory alloy such as nitinol. The chamber may be sealed with a bonding layer forming a side of the chamber opposite the sensor membrane. Where the vibratable sensor includes a bonding layer for sealing the chamber, the bonding layer may also be used for attachment to an anchoring means. In one embodiment, the bonding layer provides a hermetic seal for the chamber disposed in the vibratable sensor. The bonding layer may comprise Pyrex®, glass, silicon, or other suitable materials.

Generally, the vibratable sensor is manufactured by etching the appropriate shape and materials from a larger panel of the material. For example, the larger panel of material may be covered with a mask, the mask defining the shape of a plurality of the desired vibratable sensors, and then subjected to etching, which may be, for example, chemical etching or physical etching. The mask protects those areas of the panel that must not be removed during the etching process in order to produce the desired shape. For example, a plurality of vibratable sensors is formed when a mask having a plurality of precisely measured cut-outs cover a larger panel of material during the etching process, until chambers of the desired shape are produced in the larger panel to a depth that is substantially equal to a cut-out in the mask. The depth of the chamber may be controlled by various factors, for example where chemical etching is used: the volatility, duration, and number of chemical treatments. Each vibratable sensor may then be cut from the larger panel by slicing between consecutive chambers such that the amount of material remaining on each side of the chamber will be the thickness of walls defining a chamber in the vibratable sensor. The amount of material remaining between the bottom surface of the chamber and bottom of the larger panel will be the thickness of the sensor membrane. Any material that requires joining may be connected, for example, by brazing or welding.

As noted above, the vibratable sensor may additionally include a bonding layer of, for example, Pyrex® or other suitable material, in order to hermetically seal the vibratable sensor, preferably by joining the bonding layer to the walls of the chamber such that the bonding layer and sensor membrane are substantially parallel. In one embodiment, the bonding layer and sensor membrane form opposite walls of a vibratable sensor chamber. The bonding layer may provide a surface for attachment to anchors or other components.

Another aspect of the invention relates to a method for determining pressure in any body lumen. Once the sensor device 100 (FIG. 1) is located, data is collected using the transmitter/receiver array 103, 106 as illustrated in FIG. 1. A frequency comb 101 of acoustic beams is generated by a frequency transmitter 103, and applied (i.e., transmitted) to a passive sensor device 100. The frequency comb 101 is typically initiated by positioning the frequency transmitter 103 in close but external proximity to the sensor device 100, where “close proximity” is any distance sufficient to apply a frequency comb 101 to sensor device 100 in accordance with the devices and methods herein. When excited by the frequency comb 101, the vibratable sensor vibrates and creates modulated acoustic waves 105 (i.e., a frequency response). The modulated acoustic waves 105 are received by an ultrasound receiver 106 which is also placed in close proximity to the sensor device 100.

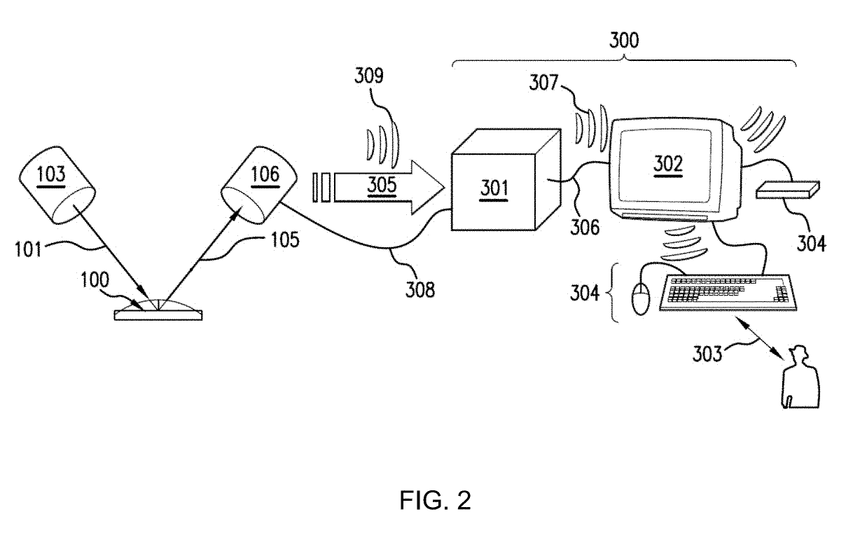

FIG. 2 shows one embodiment of a processing and display system 300 of the system of the current invention and illustrates operation of the sensor device in the system. FIG. 2 makes reference to FIG. 1, which illustrates a generic sensor device 100 of the system of the invention.

Referring to FIG. 2, ultrasound receiver 106 transmits data 305 to processing unit 301. Data 305 may include radio waves, electrical signals, digital signals, waveform signals, or any other means sufficient for communicating the acoustic properties of modulated acoustic waves 105, as received by ultrasound receiver 106. Processing unit 301 interprets data 305 using the properties of modulated acoustic waves 105 to determine a frequency response of the sensor device 100. The frequency response of the sensor is defined herein as the frequency of vibrations, including at least one resonance frequency, emitted by the sensor in response to the transmission of ultrasonic vibrations from frequency transmitter 103, at a given ambient pressure. For example, the frequency response of sensor device 100 is known when sensor device 100 is subject to “normal”, i.e., non-symptomatic, physiological conditions. The internal pressure of sensor device 100—i.e., the pressure within the cavity—is known and substantially constant. In the portal venous system, for example, the frequency response of sensor device 100 changes in accordance with changes in the venous pressure. Low-frequency acoustic waves 102, for example at 50 kHz, will stimulate at least one frequency response of vibrations in sensor device 100, at a given pressure, by exciting vibrations in vibratable sensor 2. High frequency acoustic waves, for example 750 kHz, may be used to interrogate the excited vibratable sensor. This results in modulated acoustic waves 105 that can be detected by ultrasound receiver 106.

One type of frequency response which may be measured according to the present invention is a resonance frequency. The lowest-energy resonant frequency is generally known as the fundamental frequency. Many objects have more than one resonant frequency and may vibrate at integer multiples of the resonant frequency (e.g., 2×, 3×, 4×, 5×, etc.). For example, the fundamental frequency and one or more higher order harmonic frequencies of the sensor device 100 may be identified as the frequencies which exhibit peak vibration amplitudes or relative maxima amplitudes returned from the sensor device 100.

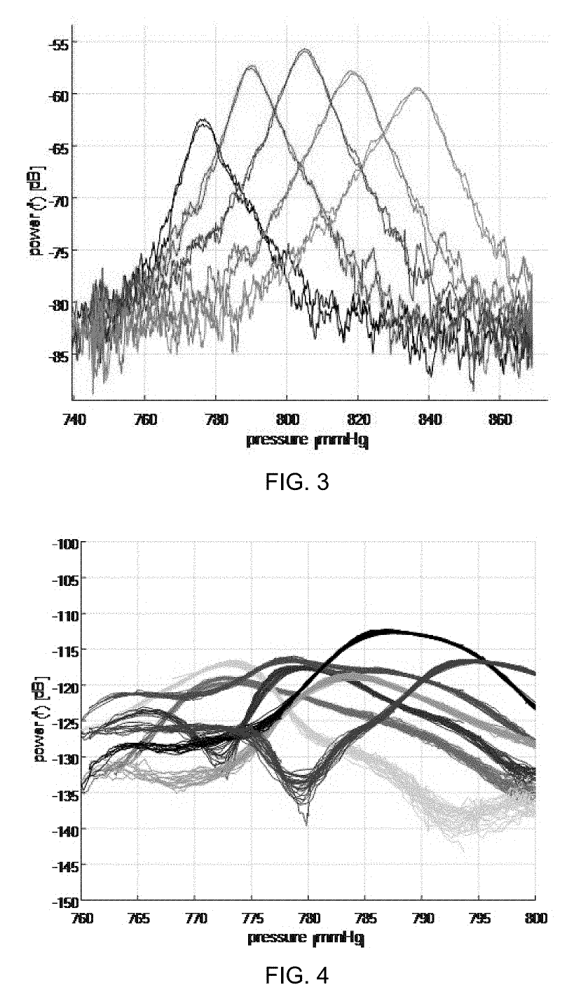

In an embodiment, a plurality of frequencies within a frequency comb of N frequencies (fᵢ i=1 . . . N) are integer multiples of the initial frequency. In the above scenario, the sensor would show an excited response at all frequencies at the comb because as a result of constructive interference of the transmitted frequencies. The result of such a response is the warping or distortion of the spectral response of the sensor in the higher harmonics which may result in a drastic reduction of system performance. To avoid this response, the frequency comb is designed using frequencies that are non-uniformly spaced and are not multiples of the resonance frequency of the resonator. FIG. 3 illustrates an exemplary spectral response of a sensor to a standard frequency comb calibrated to excite the resonant frequency of the sensor r while FIG. 4 illustrates an exemplary spectral response of a sensor to a standard frequency comb calibrated to excite the second harmonic frequency of the sensor. In particular, FIG. 3 illustrates an example of signal power (in decibels) as a function of pressure (in millimeters of mercury) where the different curves each represent a different frequency. Each peak is a resonant frequency at a different pressure. FIG. 4 illustrates an example of signal power (in decibels) as a function of pressure (in millimeters of mercury) where each curve identifies the response at a unique frequency. As can be seen in FIG. 4, no response curve has an absolute maxima. Instead, linear systems used with higher harmonics (such as the one shown in FIG. 4) include local maxima or, in extreme cases, pseudo-maxima peaks, which may provide erroneous results due to constructive interference of the transmitted frequencies thus causing erroneous results when determining ambient pressure.

A standard frequency comb is composed of a set of equally spaced frequencies, i.e., f=f₁+df*(n-1). This is not effective for detecting sensors excited at the higher harmonics (e.g., second harmonic) of the sensor device, as many objects have harmonic frequencies that are multiples of the first harmonic frequency which leads to distortion. Specifically, a receiver picks up the frequencies reflected by the sensor as well as the frequencies transmitted by the transducer. If two frequencies from the transducer added together are equal to the higher harmonic frequency of the sensor, the data will be distorted because the cause of the response will be unknown. For example, using a traditional frequency comb of 38 kHz, 39 kHz, 40, kHz, 41 kHz, and 42 kHz, if the receiver detects a large response at 80 kHz, it is possible that a sensor with a resonance frequency of 40 kHz is responding at its second harmonic, but it may also be a result of constructive interference between the 38 kHz wave and 42 kHz wave or 39 kHz wave and 41 kHz wave. This distorts the data, making it unusable. In order to overcome the distortion, a non-uniformly spaced frequency comb in which none of the frequency pairs add to double the value of a third frequency, that is, the full comb satisfies the equation fₘ+fₙ≠2f₁ wherein fₘ, fₙ, and f₁ are different frequencies within the comb. An example of a non-uniformly spaced comb is as follows:

f[kHz]=50.1, 50.5, 51, 51.6, 52.1, 52.5, 53, 53.6, 54.3, 54.6, 55.4, 55.8, 56.6, 56.9, 57.5, 57.9

The non-uniformly spaced frequency comb may be swept along a range of frequencies to detect a frequency response of the sensor (which may or may not include a resonant frequency). The sensor's calibration curve provides a range of frequencies corresponding to different pressures. The frequencies used in the comb may be chosen to correspond to a required pressure range according to the calibration curve. Because the frequency comb is non-uniformly spaced and satisfies the equation fₘ+fₙ≠2f₁ any response will be attributable to one possible cause. One way to find more possible frequency combs that fit this requirement is to shift the non-linear frequency comb by a constant frequency which maintains the properties required for this system as described above. Multiplying each frequency in the non-linear frequency comb by a constant number also maintains the required properties of the frequency comb. This allows one to adjust the non-linear frequency comb to suit different sensors or pressure ranges.

The invention may also be used with third harmonics of frequencies. In this embodiment, the frequency comb must satisfy the equation 3fᵢ≠fᵢ+fⱼ+fₖ wherein fᵢ,fⱼ, and fₖ are different frequencies. An example of such a comb is as follows:

f[kHz]=50.1, 50.5, 51, 51.6, 52.1, 52.5, 53, 53.6

As with the comb used for second harmonics, one way to find more possible frequency combs that fit this requirement is to shift the non-linear frequency comb by a constant frequency which maintains the properties required for this system as described above. Multiplying each frequency in the non-linear frequency comb by a constant number also maintains the required properties of the frequency comb. This allows one to adjust the non-linear frequency comb to suit different sensors or pressure ranges.

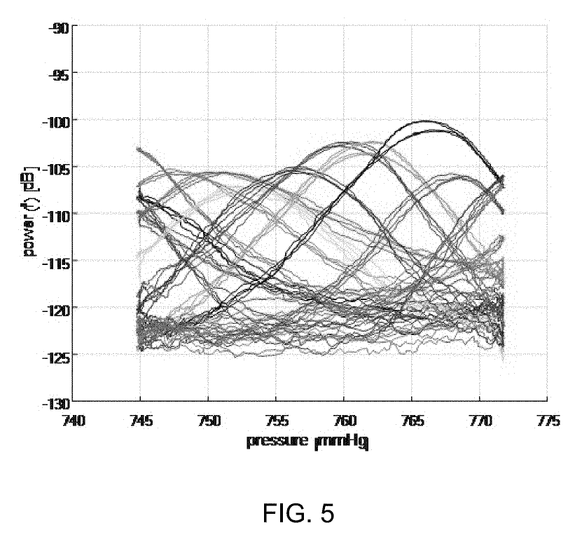

An exemplary frequency response measured using the inventive non-uniform frequency comb described above is shown in FIG. 5. FIG. 5 illustrates an example of a spectral pressure response as a function of pressure corrected by sweeping the non-uniformly spaced frequency comb along a range of frequencies. Each curve has one maximum. Given the nature of the inventive frequency comb as described above, it is easily determinable whether the maximum is a harmonic frequency of the pressure sensor or simply a reflection of waves created by the transducer on their own, or after constructive interference. This determination is clear because each maximum can only result from either one specific constructive interference or from a higher harmonic of an initial frequency. Once determined, the relevant curves can be used to determine pressure of the sensor.

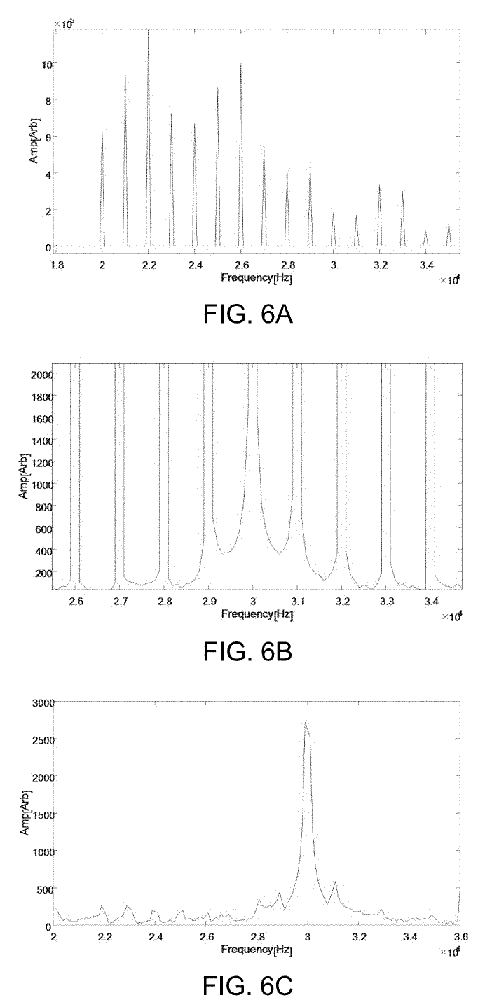

In alternative embodiments, instead of using the relative maxima and minima of the response frequencies as references points, the invention processes frequency responses in between the relative maxima and minima. The amplitude of the signal in between the relative maxima and minima is proportional to the sensor's relative change rate. The sensor's relative change rate is determined by the pressure change rate, the sensor's relative sensitivity and the sensor's quality factor, that is, the central frequency divided by the bandwidth. In one embodiment, the frequency comb must satisfy the inequality df_{comb}>df_{system} resolution in which df refers to the change in frequency. Satisfying this inequality allows measurement in frequencies which occur in the gaps between the comb frequencies. To obtain the measurements of the gap frequencies, the comb frequencies are not measured. FIG. 6A shows the full signal response of a standard linear frequency comb. FIG. 6B shows the graph of FIG. 6A, but zoomed in to show the responses between the peaks of the graph. FIG. 6C shows the signal after removal of values at transmitted frequency. These graphs show how the in-between response is masked by the response caused by the direct transmission. Because the in-between response is used in this technique, the frequency resolution must be higher than the space between the frequencies within the frequency comb. By measuring frequencies only in-between the transmitted frequencies, a useful response is attainable as shown in FIG. 6C denoting sensor response. This enables calculation of the sensor resonance frequency based on the known proportionality of the sensor. In some embodiments, this technique may be used with a linear frequency comb. In others, it may be used with a non-linear frequency comb.

The difference between the actual resonance frequency and higher order harmonic frequencies excited in the sensor device 100 and the resonance frequency and higher order harmonic frequencies of the sensor device under normal conditions is correlated to the difference in pressure between normal conditions and the actual pressure. Thus, actual pressure may be calculated based on the measured resonance frequencies of sensor device 100.

In one embodiment of the invention, the transmitter is an annular low frequency piezoelectric transducer having a working range of 0-100 kHz, 30-100 kHz, or 50-100 kHz, for example, depending on the precision required. It is, however, noted that any other suitable frequency transducer known in the art may be used for implementing the invention. In an alternate embodiment the frequency comb is made of frequencies within the range 20 KHz to 100 KHz. In another embodiments, the frequency comb moves across a range of frequencies.

In another embodiment of the invention, the frequency transmitter 103 is an annular frequency transmitting transducer, implemented as a low noise (i.e., low-range or low-bandwidth) frequency generator unit designed to generate a frequency comb of acoustic waves 101 at, for example, 750 kHz. It is noted, however, that other different values of the acoustic wave may also be used in implementing the present invention.

In one embodiment of the invention, shown for example in FIG. 2, ultrasound receiver 106 may be a disc-like high frequency receiving piezoelectric transducer. The frequency transmitter 103 and the ultrasound receiver 106 are, for example, a model CLI 7900 general-purpose ultrasonic probe, commercially available from, for example, Capistrano Labs, Inc., San Clemente, Calif., USA. When the acoustic waves including the frequency comb of acoustic waves 101 are directed at the sensor device 100, the ultrasound receiver 106 receives the modulated acoustic waves 105 which are excited in the sensor device 100 as well as other noise, e.g., signals that are reflected from other materials in the measurement environment or interference. The ultrasound receiver 106 generates an electrical signal representative of the returning acoustic signals that it receives. The electrical signal produced by the ultrasound receiver 106 is processed by the system described herein, for example as shown in FIG. 2.

In another embodiment, frequency transmitters 103 has a working range of 30-90 kHz, and transmits acoustic frequencies, for example, at 50 kHz; frequency transmitter 103 transmits, for example, at approximately 750 kHz with a narrow bandwidth (range); ultrasound receiver 106, and may, for example, operate in the range of 750 (high)±50 (low) kHz. Frequency transmitter 103, and ultrasound receiver 106 may alternatively operate in any range suitable for use with the devices and methods disclosed herein, and as particularly required for measuring fluid pressure in particular environments. In embodiments, the receiver is a wide band receiver with a bandwidth which is at least 100% of the base frequency.

The ultrasound receiver 106 is a transducer used for receiving the signals returning from the sensor when the sensor is interrogated by the frequency comb of acoustic waves 101. For example, the transducer may be implemented using suitable piezoelectric transducers. Other types of transducers known in the art may also be used to implement the transducers, such as, but not limited to, capacitive transducers, wideband capacitive transducers, composite piezoelectric transducers, electromagnetic transducers, various transducer array types, cMUTs, cymbal transducers and various suitable combinations of such transducers configured for obtaining different frequencies and/or beam shapes. For example, acoustic receivers manufactured by Vemco, PCB Piezoelectronics, and Hardy Instruments may be used.

Modulated acoustic waves 105 are the result of combining acoustic waves 101 in a reversible manner, in order to achieve a waveform with a desired frequency, wavelength, and/or amplitude. Unmodulated noise, for example caused by reflections of acoustic waves off materials in the sensor device 100 environment, is thus distinguished from the modulated acoustic waves 105 that are excited by the sensor device 100. When the received signal amplitude (in dB) is analyzed according to the frequency (in MHz), the amplitude peaks at the resonance frequency of the sensor device 100. Ultrasound receiver 106 communicates the modulated acoustic waves 105 to a processing and display system, detailed in FIG. 2, for interpretation and use.

In one embodiment, vibrations excited in sensor device 100 are distinguished from noise by correlating pressure measurements to a heart rate or pulse measurement. In this embodiment, a plurality of pressure measurements are taken during the interrogation period, for example, at least one cycle of expansion and contraction of the heart (pulse cycle). During the pulse cycle, the pressure of the entire vascular system will change continuously as the heart draws blood in and forces blood out. Accordingly, an acoustic signal that changes in a consistent manner correlated to the pulse cycle is evidenced by an excitation in the sensor. Noise reflected from, for example, surrounding tissues in the interrogation environment, does not produce such a continuously changing signal that is correlated to the pulse cycle. The above features are not limited to a single embodiment; rather, these features and functions may be applied in place of or in conjunction with other embodiments and concepts herein. The pulse cycle and waveform may be measured by an external device, for example using a pulse oximeter, heart rate monitor, ECG, etc. Optionally, such instruments may be connected to the pressure monitoring system of the invention to input the pulse or pulse waveform into the system for correlation with the acquired pressure waveform from the sensor to determine the validity of the acquired signal.

According to one aspect of the invention, the implanted sensor device 100 is subjected to a frequency comb of acoustic waves 101, the latter excites vibrations in the sensor device 100, and the reflected acoustic waves are then manifested as modulated acoustic waves 105. Ultrasound receiver 106 receives the modulated acoustic waves 105 and communicates the properties of the modulated acoustic waves 105 to a processing and display system, detailed in FIG. 2, for interpretation and use.

Returning to FIG. 2 which shows one embodiment of a processing and display system 300 of the current invention, data 305 from ultrasound receiver 106 is transmitted to a processing unit 301 which determines the pressure of the environment surrounding the sensor device 100. Data 305 is communicated between ultrasound receiver 106 and processing unit 301 via a wired 308 or wireless 309 connection. Wired connection 308 is, for example, an electronic cable or integral connection, or the like. Wireless connection 309, for example, operates by transmitting radio waves, acoustic waves, or other known media for remotely communicating data.

Processing unit 301 may comprise a computer, workstation, or other electrical or mechanical device programmed to perform the data conversions and/or displays described herein and as needed for the method of use. By way of a non-limiting example, the invention may be practiced on a standard workstation personal computer, for example those manufactured by Dell, IBM, Hewlett-Packard, or the like, and which typically include at least one processor, for example those manufactured by Intel, AMD, Texas Instruments, or the like. Processing unit 301 also comprises dedicated hardware and/or software, e.g., a data capture system such as the National Instruments PCI-6115 data capture board or may be comprised of a custom designed device for that purpose.

The output of processing unit 301 is a pressure measurement that is converted to a usable, displayable measurement either by processing unit 301 or display unit 302, or a combination thereof. For example, pressure measurements may be reported in numerical units of mmHg or Torr or maybe displayed with relation to a predefined arbitrary scale. Display unit 302 may comprise a monitor, numerical display, LCD, or other audio or visual device capable of displaying a numerical measurement. As shown in the embodiment of FIG. 2, display unit 302 is connected to or integral with processing unit 301 by connection 306, for example in the case of a computer with processing and display units, which optionally includes as a remote element, separate wired element, or integral element to processing 301 and/or display units 302, interface 303 and input/output elements 304, such as a keyboard, mouse, disk drive, optical pen, or the like, to allow a user to collect, manipulate, track, and record data. Connection 306 may optionally be a remote connection 307, operating by transmission of radio waves, acoustic waves, or other known remote transmission methods.

It will be appreciated by persons having ordinary skill in the art that many variations, additions, modifications, and other applications may be made to what has been particularly shown and described herein by way of embodiments, without departing from the spirit or scope of the invention. Therefore, it is intended that the scope of the invention, as defined by the claims below, includes all foreseeable variations, additions, modifications, or applications.

What is claimed is:

1. A system for detecting a resonance frequency comprising,
a passive mechanical resonator;
an ultrasonic transducer;
a receiver, said receiver coupled to a processer;
said ultrasonic transducer configured to generate a signal, said signal comprising a frequency comb having a plurality of non-uniformly spaced frequencies,
said receiver receiving a modulated acoustic signal, and;
said processor, processing the modulated acoustic signal to determine a resonance frequency.

2. The system of claim 1 wherein, said frequency comb is moved across a range of frequencies.

3. The system of claim 1 wherein, said plurality of non-uniformly spaced frequencies is within the range of 20 KHz-100 KHz.

4. The system of claim 1 wherein, the sum of said plurality of non-uniformly spaced frequencies are not multiples of the resonance frequency of said resonator.

5. The system of claim 3 wherein, said receiver is a wide band receiver with a bandwidth which is at least 100% of the base frequency.

6. A method for determining a resonant frequency using a passive mechanical resonator, said method comprising the following steps:
generating an acoustic signal, said acoustic signal comprising a frequency comb having a plurality of non-uniformly spaced frequencies,
receiving a modulated acoustic signal,
processing the modulated acoustic signal to determine a resonance frequency.

7. The method of claim 6 further comprising, shifting said frequency comb a range of frequencies.

8. The method of claim 6 wherein, said plurality of non-uniformly spaced frequencies is within the range of 20 KHz-100 KHz.

9. The method of claim 6 wherein, said plurality of non-uniformly spaced frequencies are not multiples of the resonance frequency of said resonator.

10. A method for determining a resonant frequency using a passive mechanical resonator, said method comprising the following steps:
generating an acoustic signal, said acoustic signal comprising a frequency comb having a plurality of frequencies,
receiving a modulated acoustic signal,
subtracting the frequency comb from said returning signal, and;
processing the modulated acoustic signal to determine a resonance frequency.

11. The method of claim 16 further comprising, shifting said frequency comb a range of frequencies.

12. The method of claim 16 wherein, said plurality of frequencies is within the range of 20 KHz-100 KHz.
